(19) United States
(12) Patent Application Publication
(10) Pub. No.: US 2022/0213511 A1
(43) Pub. Date: Jul. 7, 2022

(54) ANAEROBIC DIGESTER AND MOBILE BIOGAS PROCESSING PLANT

(71) Applicant: BENNAMANN SERVICES LTD, Newquay, Cornwall (GB)

(72) Inventors: Christopher MANN, St. Mawgan, Cornwall (GB); Matthew John HAGUE, Ampthill, Bedfordshire (GB); Thomas William BRADSHAW, Wantage, Oxon (GB)

(73) Assignee: BENNAMANN SERVICES LTD, Newquay, Cornwall (GB)

(21) Appl. No.: 17/609,726

(22) PCT Filed: May 8, 2020
(86) PCT No.: PCT/IB2020/054392

§ 371 Date: Nov. 8, 2021

Related U.S. Application Data
(60) Provisional application No. 62/845,689, filed on May 9, 2019.

(51) Int. Cl. C12P 5/02; C12N 1/20; C12M 1/107; C12M 1/00; C10L 3/10; F25J 3/06; F17C 1/00

(52) U.S. Cl. CPC C12P 5/023; F17C 2223/036; C12M 21/04; C12M 23/36; C12M 47/18; C12M 29/04; C12M 23/52; C10L 3/104; F25J 3/061; F25J 3/0635; F25J 3/067; F17C 1/00; C10L 2290/548; C10L 2290/26; C10L 2290/06; C10L 2290/567; F17C 2201/0185; F17C 2221/033; C12N 1/20

(57) ABSTRACT

An anaerobic digester is provided. The anaerobic digester includes a biogas storage container comprising a semi-permeable membrane separating the biogas storage container into a first space and a second space, such that the first space is configured to be methane enriched and the second space is configured to be CO₂ enriched. The anaerobic digester further includes a cover positioned over the biogas storage container for protecting the biogas storage container against the elements.

### FIELD OF THE DISCLOSURE

This disclosure relates generally to anaerobic digestion and biogas processing.

### BACKGROUND

Anaerobic digestion is a process that can be used to convert a wide range of biomass materials into methane and carbon dioxide gases. Carbon dioxide can be used for a variety of purposes such as food and industrial processing. Methane, which is typically more valuable than carbon dioxide, can be used as a direct replacement for fossil fuels such as oil and natural gas. When methane is generated from anaerobic digestion from organic matter (i.e. biomass), it is often referred to as biomethane.

Biomethane can be used as a fuel (e.g., for combustion engines or fuel cells) to provide power and heat. When biomethane is burnt, the exhaust comprises only carbon dioxide and water. In principle, the quantity of carbon dioxide released equals the amount that would have been released had the biomass had been allowed to aerobically decompose naturally; therefore, methane produced in this way is effectively considered a zero-carbon fuel. The use of anaerobic digestion of biomass to produce methane is therefore seen as an effective way to reduce the level of carbon dioxide in the atmosphere and help to mitigate climate change.

Methane itself is a more potent greenhouse gas than carbon dioxide. Over 20 years, 1 kg of methane equates to 86 kg of carbon dioxide, while over 100 years the same 1 kg of methane equates to 25 kg of carbon dioxide. It is therefore important to prevent leakage of methane during the anaerobic digestion process (otherwise it would not be a zero-carbon fuel). Accordingly, related art has been developed to improve the use and storage of methane during anaerobic digestion and ensuring that there is no leakage of methane.

However, related art anaerobic digestion systems and methods may not be feasible on small- to mid-sized farms (e.g. dairy farms having from about 50 to about 100 cows). While such farms produce more methane than they need to maintain their operations, it is not always economical to invest in large installations for managing anaerobic digestion on such scales.

Accordingly, there is a need for improvements to anaerobic digestion systems and methods.

### SUMMARY

According to embodiments, an anaerobic digester is provided. The anaerobic digester includes, for instance, a biogas storage container comprising a semi-permeable membrane separating the biogas storage container into a first space and a second space, such that the first space is configured to be methane enriched and the second space is configured to be CO₂ enriched. In certain aspects, the anaerobic digester further includes a cover positioned over the biogas storage container for protecting the biogas storage container against the elements.

In some embodiments, the anaerobic digester further includes a biomass storage container configured to contain biomass; a biomass input channel coupled to the biomass storage container for providing biomass to the biomass storage container; a first output valve coupled to the first space of the biogas storage container; and a second output valve coupled to the second space of the biogas storage container. In some embodiments, the semi-permeable membrane comprises a stretched polytetrafluoroethylene-based material or silicone. In some embodiments, the cover positioned over the biogas storage container is transparent, and is configured to provide passive solar heating to the biogas storage container.

In some embodiments, the anaerobic digester further includes one or more sensors for measuring one or more parameters, and an apparatus capable of communicating wirelessly with a remote site to transmit the one or more parameters. In some embodiments, the one or more parameters includes at least one of an amount of biogas and a concentration of biogas.

According to another aspect, a method for installing an anaerobic digester according to any one of the embodiments of the first aspect is provided. The method can include, for instance, digging a pit to construct a space for storing biomass for use in the anaerobic digester. The method may further include installing the biogas storage container at least partially in the pit. The method further includes installing the cover positioned over the biogas storage container for protecting the biogas storage container against the elements.

According to embodiments, a method for using an anaerobic digester described herein is provided. The method can include feeding biomass into an anaerobic digester via a biomass input channel, as well as extracting enriched biogas from the digester.

According to embodiments, a mobile biogas processing plant is provided. In certain aspects, the mobile biogas processing plant can be adapted to process one or more enriched gases as inputs. For instance, the mobile biogas processing plant can include a CO₂ input for receiving CO₂-enriched gas. The mobile biogas processing plant can further include a methane input for receiving methane-enriched gas. The mobile biogas processing plant may have a removal stage (e.g., CO₂ removal) and a liquefaction stage (e.g., methane liquefaction). In some embodiments, the two stages may be combined into a single stage. Additional cooling elements, such as heat exchangers, refrigeration units, and sacrificial cryogenic liquids may also be used in one or more of the stages.

In some embodiments, the mobile biogas processing plant comprises one or more temperature regulation components. In certain aspects, the mobile biogas processing plant further includes a compressor coupled to the methane input and configured to compress methane gas. For instance, according to some embodiments, a mobile biogas processing plant further includes a first heat exchanger coupled to the compressor. The mobile biogas processing plant can further include a cold box coupled to the first heat exchanger, wherein the cold box is configured to liquefy CO₂ gas but not methane gas. While removal of CO₂ as a liquid is used as an example, CO₂ may also be removed as a solid according to some embodiments. In some embodiments, the mobile biogas processing plant further includes a Joule Thompson unit or cryocooler coupled to the cold box, wherein the Joule Thompson unit or cryocooler is configured to liquefy methane gas. While a Joule Thompson unit and cryocooler are used as examples, other refrigeration and cooling devices may be employed in some instances.

In some embodiments, the mobile biogas processing plant is transportable to a remote site, and the methane output is removable and replaceable such that the methane output (e.g., storage container) can be removed and replaced when full or nearly full with a replacement unit. In some embodiments, the CO₂ and methane inputs are coupled to an anaerobic digester according to any one of the embodiments herein. In some embodiments, the compressor is configured to be powered by CO₂-enriched gas received from the CO₂ input.

According to embodiments, a method for using a mobile biogas processing plant described herein is provided. The method optionally includes compressing a biogas mixture to a processing pressure. The method further optionally includes feeding the biogas mixture into a first heat exchanger. The method can also include feeding the biogas mixture from the first heat exchanger into a cold box, wherein the biogas mixture is cooled, thereby causing substantially all the carbon dioxide in the biogas mixture to drop (e.g., as a liquid) to a bottom of the cold box. The method further optionally includes feeding the liquefied carbon dioxide from the cold box into the first heat exchanger. The method further includes feeding the remaining biogas mixture from the cold box into the Joule Thompson unit or cryocooler, wherein the remaining biogas mixture is cooled thereby causing a first portion of the methane in the remaining biogas mixture to liquefy. Sacrificial liquid cryogens may also be used as part of removal or liquefaction.

In some embodiments, the processing pressure is in a range of from about 100 bar to about 300 bar. In some embodiments, the biogas mixture prior to being fed into the first heat exchanger is about 85% methane and about 15% carbon dioxide.

According to embodiments, a node for managing one or more anaerobic digesters is provided. The node includes a processor and circuitry configured to receive input from the anaerobic digesters regarding an amount of biogas for each of the anaerobic digesters. Where the node is connected to more than one digester, the processor and circuitry are further configured to select one of the plurality of anaerobic digesters for processing based at least in part on the amount of biogas for each of the plurality of anaerobic digesters. The processor and circuitry are further configured to schedule delivery of mobile biogas processing plant to the selected one of the plurality of anaerobic digesters.

In some embodiments, the processor and circuitry are further configured to receive pricing information indicating a price of biogas or another fuel; and receive energy level information indicating an energy level of a distribution site. Selecting one of the plurality of anaerobic digesters for processing can be further based on the pricing information and energy level information. In some embodiments, selecting one of the plurality of anaerobic digesters for processing comprises selecting a set of anaerobic digesters of the plurality of anaerobic digesters, and ordering the set of anaerobic digesters based at least in part on the amount of biogas for each of the plurality of anaerobic digesters.

According to embodiments, a method for managing one or more anaerobic digesters is provided. The method includes receiving input from the anaerobic digesters regarding an amount of biogas for each of the anaerobic digesters. Where more than one digester is monitored, the method further includes selecting one of the plurality of anaerobic digesters for processing based at least in part on the amount of biogas for each of the plurality of anaerobic digesters. The method further includes scheduling delivery of mobile biogas processing plant to the selected one of the plurality of anaerobic digesters.

In some embodiments, the method further includes processing biogas (e.g., enriched biogas) from at least one digester (e.g., the selected one of the plurality of anaerobic digesters) by the mobile biogas processing plant.

Other features and characteristics of the subject matter of this disclosure, as well as the methods of operation, functions of related elements of structure and the combination of parts, and economies of manufacture, will become more apparent upon consideration of the following description and the appended claims with reference to the accompanying drawings, all of which form a part of this specification, wherein like reference numerals designate corresponding parts in the various figures.

### BRIEF DESCRIPTION OF THE DRAWINGS

The accompanying drawings, which are incorporated herein and form part of the specification, illustrate various embodiments of the subject matter of this disclosure. In the drawings, like reference numbers indicate identical or functionally similar elements.

FIG. 1 illustrate an exemplary anaerobic digester according to some embodiments.

FIG. 1A illustrates an exemplary biogas storage container according to some embodiments.

FIG. 2 illustrates an exemplary biogas separation and methane liquefier according to some embodiments.

FIG. 2A illustrates an exemplary CO₂ removal unit (e.g., cold box) according to some embodiments.

FIG. 2B illustrates an exemplary liquefaction unit (e.g., Joule-Thompson unit) according to some embodiments.

FIG. 2C illustrates an exemplary combination CO₂ removal and liquefaction unit according to some embodiments.

FIG. 3 illustrates an exemplary system according to some embodiments.

FIG. 4 illustrates a flow chart according to some embodiments.

FIG. 5 illustrates a block diagram of an apparatus (such as associated with an anaerobic digester, mobile biogas processing plant, or logistics coordination center) according to some embodiments.

FIG. 6 illustrates phase diagrams for carbon dioxide and methane.

FIG. 7 is a table illustrating material permeability.

### DETAILED DESCRIPTION

While aspects of the subject matter of the present disclosure may be embodied in a variety of forms, the following description and accompanying drawings are merely intended to disclose some of these forms as specific examples of the subject matter. Accordingly, the subject matter of this disclosure is not intended to be limited to the forms or embodiments so described and illustrated.

Unless defined otherwise, all terms of art, notations and other technical terms or terminology used herein have the same meaning as is commonly understood by persons of ordinary skill in the art to which this disclosure belongs. All patents, applications, published applications and other publications referred to herein are incorporated by reference in their entirety. If a definition set forth in this section is contrary to or otherwise inconsistent with a definition set forth in the patents, applications, published applications, and other publications that are herein incorporated by reference, the definition set forth in this section prevails over the definition that is incorporated herein by reference.

Unless otherwise indicated or the context suggests otherwise, as used herein, “a” or “an” means “at least one” or “one or more.”

This description may use relative spatial and/or orientation terms in describing the position and/or orientation of a component, apparatus, location, feature, or a portion thereof. Unless specifically stated, or otherwise dictated by the context of the description, such terms, including, without limitation, top, bottom, above, below, under, on top of, upper, lower, left of, right of, in front of, behind, next to, adjacent, between, horizontal, vertical, diagonal, longitudinal, transverse, radial, axial, etc., are used for convenience in referring to such component, apparatus, location, feature, or a portion thereof in the drawings and are not intended to be limiting.

Furthermore, unless otherwise stated, any specific dimensions mentioned in this description are merely representative of an exemplary implementation of a device embodying aspects of the disclosure and are not intended to be limiting.

As used herein, the term “adjacent” refers to being near or adjoining. Adjacent objects can be spaced apart from one another or can be in actual or direct contact with one another. In some instances, adjacent objects can be coupled to one another or can be formed integrally with one another.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the embodiments described herein.

Many types of biomass can be anaerobically digested. To achieve the most beneficial impact with respect to climate change, using anaerobic digestion to limit or eradicate “fugitive” emissions of methane (such as those that are currently created by the poor management of animal manures such as cow and pig slurry in open lagoons) may be most effective. The use of open-slurry lagoons in the agriculture sector results in very high levels of fugitive methane emissions. By sealing the slurry lagoon to prevent aerobic digestion, the methane can be contained. This practice can be advantageous for the purposes of limiting or eradicating “fugitive” emissions of methane, and in embodiments disclosed herein can also provide for considerable operational benefits. Such benefits may include:
Reduced nitrogen loss; this is because nitrogen is contained in the digestate (i.e. the material remaining after the anaerobic digestion of the biomass), which can in turn reduce the need for fertilizer when the digestate is spread back onto the land.
Reduced handling and management of slurry; this is because rain water is prevented from entering the covered lagoon, meaning that the digestate is more concentrated and there is less to spread.
Reduced risk of overspill; this is because rain water is prevented from entering the covered lagoon, and in turn minimizes the possibility of leakage of raw slurry into waterways (which is illegal in many countries).
Reduced greenhouse gases; this is because biomass (such as waste or spoiled animal feed) is usually managed by composting aerobically and the energy held in it is lost as heat during this process, and may result in large quantities of methane and nitrous oxides, both powerful greenhouse gases. Such greenhouse gases are reduced, however, with the use of a sealed slurry lagoon, such as provided by embodiments disclosed herein.
Reduced energy demands; this is because anaerobically generated methane may be used as fuel for a generator, e.g. to generate electricity and heat that can be used on the farm, thereby offsetting its electricity and energy usage.

Where the installation cost of a covered slurry lagoon is kept low, the above benefits can provide a reasonable return on investment for small- to mid-sized farms as compared to an open slurry lagoon.

The quantity of biomethane produced through the use of a covered slurry lagoon anaerobic digester varies, e.g. depending on the efficiency of the process. Typically, however, a 50- to 100-cow dairy farm can produce approximately 10-15 times more methane than it requires for its operations for heat and power. The excess methane can be used to generate power and be injected into the electricity grid or alternatively processed and upgraded for injection into the mains gas grid. In related art systems and methods, however, the investment required for such installations means that this approach is economically feasible only for large-scale farms, and even then only with government subsidies (which can be withdrawn or reduced). Therefore, the zero-carbon energy that could be recovered from the abundant source of biomass (such as animal manure slurry and waste) that is held on small, remote farms is effectively land-locked. Such farms represent the majority of total farms for most developed and developing regions. Accordingly, there is a need for exploiting the methane that a small, remote farm could generate through the anaerobic digestion of its waste and other available biomass.

Another abundant source of biomass is grass cuttings, such as on managed land including gardens, sports fields, roadside verges, and golf courses. Currently, either the grass cuttings are left where they fall or are collected and composted. Either way, this is carried out aerobically, thereby losing the methane generation potential as wasted heat. In addition, much of the northern hemisphere is covered in vast areas of unmanaged or under-utilized grassland that could be used to generate biomethane. For instance, if the grass cuttings collected from one square meter over a year were anaerobically digested, then the methane produced would have the equivalent energy of one liter of petrol. As is the case with small, remote farms, however, it is difficult to realize the true value of methane producible through this process, due to e.g. the remote location of manage or unmanaged grasslands, or the lack of electricity or gas-grid infrastructure. The financial and environmental value of this abundant form of renewable and zero carbon energy cannot be economically realized, leaving it effectively land-locked. Accordingly, there is a need for exploiting the methane that such grasslands could generate through the anaerobic digestion of its grass cuttings and other available biomass.

Embodiments provided herein disclose an anaerobic digester. Embodiments also disclose a mobile biogas processing plant and liquid methane storage unit. Some embodiments combine aspects of the anaerobic digester with the mobile biogas processing plant and liquid methane storage unit, which in combination provide additional benefits, e.g. improving the economic incentives for small- to mid-sized farms to invest in anaerobic digestion. Additionally, embodiments disclose logistical solutions for managing multiple installations of such anaerobic digesters and managing the distribution of one or more mobile biogas processing plants among the multiple installations.

U.S. Pat. No. 9,927,067 (referred to here as the Mann Patent) discloses liquid methane systems and methods, including systems and methods for storing and processing biogas, such as may be applicable to embodiments disclosed herein. Application PCT/IB2018/059148 discloses liquid methane storage and fuel delivery systems, including for example an improved cryogenic storage tank, such as may be applicable to embodiments disclosed herein. For example, the mobile biogas processing plants described herein may utilize such cryogenic storage tanks. Application PCT/IB2018/053821 discloses methods and systems for determining quality of a fuel, including for example apparatus for measuring properties of a gas or fluid flowing in a feed pipe, such as may be applicable to embodiments disclosed herein. For example, the anaerobic digesters or mobile biogas processing plants described herein may utilize such apparatus to measure properties of a gas or fluid.

Anaerobic digestion of biomass produces biogas, which is made up of four main components: water, methane, carbon dioxide, and hydrogen sulfide. In order to realize the full potential monetary value of the biogas, it must be processed to separate out the main gases that have value (methane and carbon dioxide) from the other components. Hydrogen sulfide usually makes up less than 0.5% of the resulting biogas; it is highly toxic and corrosive, and has little economic value. The simplest way of removing it is to pass it through activated carbon filters. These filters eventually become saturated and may be replaced, allowing the sulphur and carbon to be returned to the land and thereby maintaining the soils' sulphur levels and sequestering the carbon. The water can be removed using a simple refrigerated dryer.

Having removed the hydrogen sulfide and water, the methane and carbon dioxide are left. For efficient processing of these gases, the two gases may be separated. According to embodiments, there are several ways that this can be achieved. Once the gases are separated, the resulting gases need to be removed. This could involve, for instance, removal of the gases from a remote site. Both gases (methane and carbon dioxide) can be converted into their liquid form, thereby resulting in a volume reduction factor of 600. This volume reduction enables the gases to be removed (e.g. from a remote site), e.g. by road tanker. The road tanker, or other vehicle, can itself be powered by the biomethane that is being removed, rendering the whole process zero carbon.

Methane is a cryogenic gas, and therefore needs to be kept below its critical temperature using cryogenic techniques. A new zero venting liquid storage system is the subject of U.S. Pat. No. 9,927,067 (referred to hereinafter as “the Mann Patent”), the subject matter of which is incorporated herein in its entirety. Embodiments may make use of the zero venting liquid storage system disclosed in the referenced U.S. Patent, as well as other techniques for keeping methane below its critical temperature. Carbon dioxide, on the other hand, is not a cryogenic gas, and therefore can be pressurized to become a liquid or solid at ambient temperatures. Therefore, carbon dioxide may also be transported by tanker and road from a remote location where it has been generated.

The conversion of methane and carbon dioxide to their non-gaseous form, and their subsequent removal (such as through the use of a road tanker) provides a way to capture the biogas potential of small- to mid-sized farms or other remote locations. The biomass and the resulting methane and carbon dioxide from these remote areas can then be fully exploited. Cryogenic gas processing and subsequent liquefaction, however, is most economically carried out at industrial scale. The investment required for a small- to mid-sized farm to have a dedicated biogas processing and liquefaction facility would be prohibitively expensive, especially as additional investment would also be required for the cost of installing the covered slurry lagoon anaerobic digester. Small and mid-sized farms are unlikely to produce enough biogas to make such investments economical. Embodiments provide a solution to this problem by providing a mobile biogas processing plant and liquid methane storage unit.

An anaerobic digester is now described.

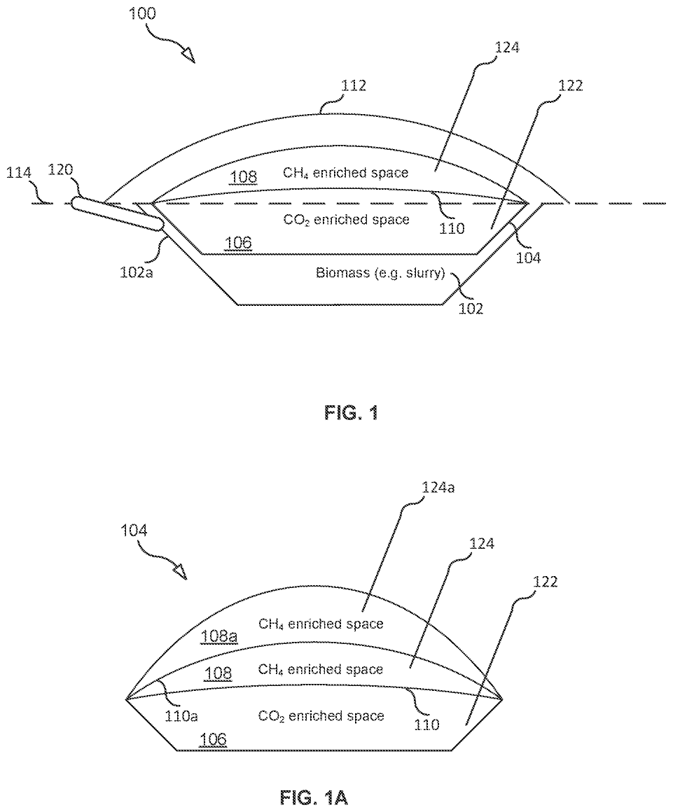

FIG. 1 illustrates an exemplary anaerobic digester 100 according to some embodiments. FIG. 1A illustrates an exemplary biogas storage container 104 according to some embodiments. Anaerobic digester 100 will now be described with reference to FIGS. 1 and 1A. Anaerobic digester 100 may take the form of a sealed slurry lagoon. As shown, anaerobic digester 100 includes a biomass storage container 102 for storing biomass (such as slurry) and a biogas storage container 104 for storing biogas resulting from the anaerobic digestion of biomass. The biogas storage container 104 is separated by a semi-permeable membrane 110 into a first space 108 and a second space 106. The membrane may be, for instance, selectively permeable to methane vs. CO₂, allowing one to pass and the other not to pass (or pass more slowly). As shown, the second space 106 is CO₂ (carbon dioxide) enriched, while the first space 108 is CH₄ (methane) enriched.

Anaerobic digester 100 may also include a cover 112, which may be positioned over the biogas storage container 104 in order to protect the biogas storage container 104 from the elements. This could include, for instance, protection from rain and/or wind. Cover 112 may be transparent, and may also provide for passive solar heating of the biogas storage container 104. It should be strong, chemically inert and immune to damage from ultraviolet light. An example may include Ethylene Tera Fluoro Ethylene (ETFE), though other materials may be suitable.

The second space 106 of the biogas storage container 104 and the biomass storage container 102 may be coextensive. That is, in some embodiments, there may be no physical separation between the biomass storage container 102 and the second space 106 of the biogas storage container 104.

Anaerobic digester 100 may also include an input 120 for receiving biomass (such as slurry) into the biomass storage container 102. Additionally, anaerobic digester 100 may include output valves 122 and 124, coupled respectively to the second space 106 and first space 108 of the biogas storage container 104. That is, the biogas located within the biogas storage container 104 may be removed from the biogas storage container 104 by pipes or hoses connecting to one or more of output valves 122 and 124. Such pipes and hoses may connect to a mobile processing plant, and provide enriched biogas to such a processing plant.

The anaerobic digester 100 may be installed underground, or partially underground. As shown, ground level 114 is indicated in FIG. 1 by a dashed line. In the embodiment shown, the biomass storage container 102 is entirely underground, the second space 106 of the biogas storage container 104 is partially underground and partially above ground, and the first space 108 of the biogas storage container 104 is above ground. Other configurations are possible. Anaerobic digester 100 may be installed by digging a pit in the ground. The pit may include inclined banks 102a. In some embodiments, there may be additional layers, such as an insulation layer provided between the ground and the biomass storage container 102. For instance, an insulation layer could prevent the slurry or other biomass from seeping into the ground, or prevent the biogas from escaping the anaerobic digester 100.

The anaerobic digester 100 may be advantageously used with existing anaerobic digesting systems (such as those described in the Mann Patent). The digester 100 also provides additional benefits to small- to mid-sized farms in that they can economically employ anaerobic digestion where before it was not feasible. Because such farms may be in remote and diverse geographic locations, it is important that in some embodiments anaerobic digester 100 is a simple design that can be readily implemented at such sites, with readily available equipment and processes. Rather than using an enclosure above ground (as is standard industrial practice), the digester 100 may be made up of an excavated hole with banks made from the removed earth. This can negate the need for removal of soil from the site and can also negate the need for concrete.

Furthermore, and according to some embodiments, by tapering the walls at an angle, the space above the digester 100 is increased so that it can hold sufficient quantity of biogas to maintain the needs of the periodic biogas processing and collection service in accordance with the space required for biogas generation from the slurry from a defined number of cows. That is, a mobile biogas processing plant may service a remote farm employing anaerobic digester 100, and may make periodic visits, or visits at aperiodic intervals. Accordingly, the digester 100 should have enough space for storing biogas that is expected to be generated before a next arrival time of the mobile biogas processing plant. Tapering the walls at an angle is one way to maximize the available space.

Anaerobic digester 100 may additionally include one or more sensors. For instance, digester 100 may include one or more sensors for measuring an amount of biomass present in the digester 100, an amount of biogas present in the digester 100, an amount of biogas present in each of the spaces (e.g. spaces 106, 108, 108a) of the biogas storage container 104, and/or a concentration of biogas present in the digester 100 or in each of the spaces of the biogas storage container 104. Such sensors may also include sensors to measure pressure and other process variables relevant to managing the biogas, such as detection of impurities. According to embodiments, the measurements are made with sensors using one or more sound waves. In certain aspects, the measurements are based on the measured speed of one or more sound waves. For instance, anaerobic digester 100 may include a first transducer coupled to one or more of the digester's gas-containing regions (e.g., 106, 108, 108a and/or feed pipes and hoses associated with the digester), where the transducer is configured to generate a first sound wave passing in a first direction through the gas. There may also be a second transducer coupled to the system to receive the first sound wave passing in the first direction through the gas or fluid flowing along the feed pipe. There may also be timing-circuitry in electrical communication with the first and second transducers and configured to measure a velocity of the first sound wave passing in the first direction from the first transducer to the second transducer. This can indicate one or more of amount of biogas, quality of biogas, etc.

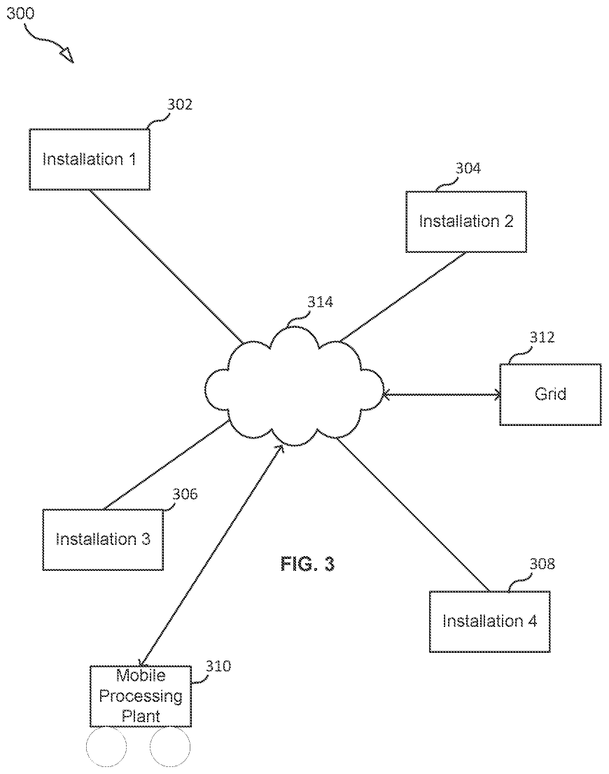

Anaerobic digester 100 may also include circuitry and other equipment in order to be able to communicate (e.g. wirelessly communicate) such sensor readings to remote sites, including the logistics coordination center 314 discussed below with reference to FIG. 3. Anaerobic digester 100 may also be able to receive commands and/or configuration settings from a remote site, including the logistics coordination center 314, that cause anaerobic digester 100 to perform some action such as adjusting a setting or configuration of the digester 100.

Biogas produced by anaerobically digesting the slurry can be pre-processed prior to liquefaction. In embodiments, such processing may include cleaning, such as by passing the biogas through a hydrogen sulfide removal system (such as activated charcoal filters). These filters may be located on the mobile biogas processing plant, implemented by one or more membranes of the anaerobic digester 100, or may be fixed installations nearby the anaerobic digester 100. The cleaning can further include drying to remove water vapor (such as by using an industrial dryer). The biogas can be passed from the anaerobic digester to the cleaning components due to the pressure generated in the anaerobic digester 100. According to embodiments, the digester (100) is maintained at positive pressure relative to the outside space as a result of the digestion of biomass in container 102. A low-pressure return valve (located on one or both ends of the connection coupling the anaerobic digester 100 and the filters for processing the biogas) ensures one-way passage through the system. This negates the need for a compressor in the processing/cleaning component, whereas in typical industrial-scale biogas cleaning and upgrading, the use of a compressor is required to force the biogas through the cleaning circuit and also through permeable gas membrane filters which are tightly packaged. A compressor like that used in such systems increases complexity and cost, and also requires additional power, which would lower the overall efficiency of the process. Accordingly, in embodiments anaerobic digester 100 advantageously does not include a compressor.

The biogas storage container 104, as described above, is separated into two spaces (106 and 108) by a semi-permeable membrane 110 in FIG. 1. The semi-permeable membrane 110 may comprise, for example, polytetrafluoroethylene (e.g., expanded PTFE (ePTFE), often referred to as Gore-Tex or Teflon) or silicone. This membrane material is selected to preferentially pass either methane or carbon-dioxide, for instance, where methane is lighter than carbon dioxide and is also a relatively large molecule compared with carbon dioxide. Other materials may be selected to exploit other variations between methane and carbon dioxide molecules.

As shown in FIG. 1, the second space 108 is positioned higher than the second space 106. A valve may be positioned to cause biogas from the slurry to move into one of the upper spaces (e.g., by pumping or natural pressurization), such as second space 108. One or more hoses or pipes may also be used. This can be beneficial because gravity can aid in the diffusion or filtering process, helping to make the second space 106 more carbon dioxide enriched and the first space 108 more methane enriched. In some embodiments, there may be additional membranes creating more than two spaces in the biogas storage container 104. For example, there may be a second membrane 110a (see FIG. 1A), and the first and second membrane would then separate the container 104 into three spaces, first space 108, second space 106, and an additional third space 108a that would contain more pure methane than the first space 108. In some embodiments, biogas from the space 102 may be fed to a middle space (e.g., space 108 in FIG. 1A). According to embodiments, a space in the digester 100 can serve as a receiving space.

While two or three spaces are used as an example in some embodiments, there may be more. For instance, there may be more than two membranes (e.g. from three to ten membranes, and even more than that in some embodiments), and thus more than three spaces (e.g., from 4-11). Different membranes may have differing properties to control the relative flow of enriched gas. For instance, differing membranes may be used to filter different materials. For instance a first membrane may selectively filter carbon dioxide, a second membrane may selectively filter water, a third membrane may selectively filter hydrogen sulfide, and a fourth membrane may selective filter water. Thus, and according to embodiments, a digester with 1, 2, 3, or 4 membranes is provides. Similarly, and according to embodiments, a digester with 2, 3, 4, or 5 spaces is provided. According to some embodiments, one or more pipes, hoses, and valves may be used to direct (e.g., by pumping or natural pressurization) the unfiltered-gas to a selected space to start the filtration process.

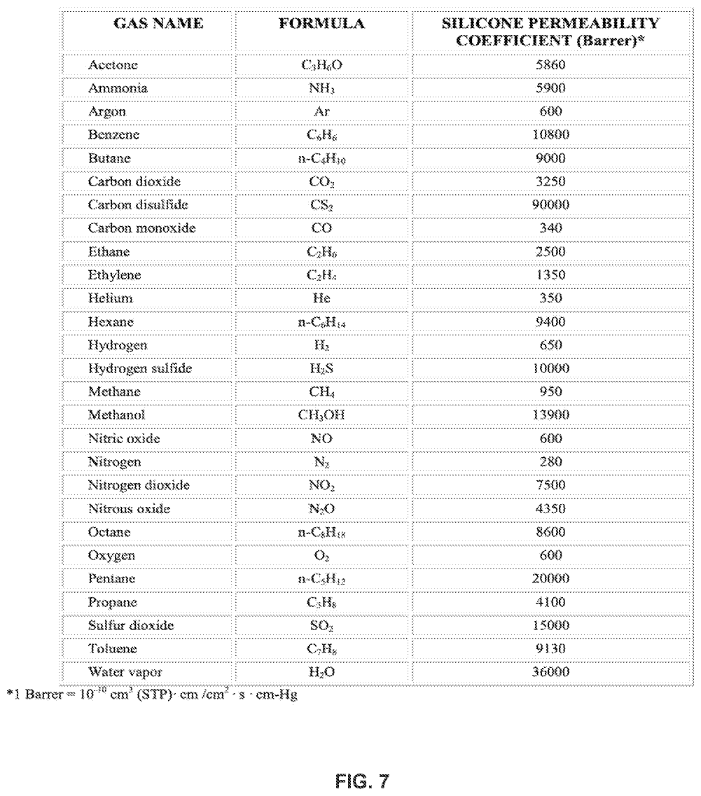

By stacking multiple spaces on top of one another, and through the use of multiple semi-permeable membranes, the diffusion process may be improved. For instance, methane may slowly progress upwards through the (vertically stacked) spaces while CO₂ moves progressively down, e.g. due to the differences in density and molecule size, and therefore the purity of the methane may increase as a function of both height and time. In such embodiments, there may also be additional valves (such as valve 124a) for removing the methane from the additional spaces, as it can be more efficient to process methane that is more pure. Also, it may be beneficial to have a relatively leaky membrane closer to the bottom and less leaky closer to the top to allow more of the heavier larger CO₂ molecules to drop down. The permeability of the various semi-permeable membranes may differ (e.g. they may have different pore sizes), and for example, could be optimized for gas production or methane purity. In some embodiments, a membrane used to selectively filter a material from methane may have a separation factor of between 3.0 and 40 depending on material (e.g., 3.42 for CO₂, 10.5 for hydrogen sulfide, and 37.9 for water). Similarly, there may be relative separation factors between the filtered materials (e.g., 3.6 between water and hydrogen sulfide and 11.7 between water and CO₂). Such selective permeability can be used advantageously in a stacked arrangement for a digester 100. A gas may become more refined when passing through different spaces and membranes. A table of permeability values for silicone is provided in Membrane Gas Exchange, by J. Patrick Montoya and MedArray, Inc. (2010), and is reproduced in FIG. 7.

In some embodiments, a composite membrane may be used, in which two or more membranes are combined. This can be used, for example, to add additional filtering properties (e.g., where the second membrane also selectively filters) or for improved strength and control of the membrane. For instance, a filtering membrane may be layered onto a backing material to add rigidity or support. According to an embodiment, the physical strength required to be self-supporting over a large area may be improved with a composite membrane, where the strength and porosity of a particular material (e.g., a aramid or para-aramid material such as Kevlar or PTFE-based material such as Gore-tex) is used as a backing material to a highly-selective silicone membrane to control the stretching of the silicone pores, which might change the properties of the membrane. Feed and permeate pressures can affect permeation rates as the membrane structure can change under pressure. For example, if the membrane is stretched, then the pore size may change leading to a change in the separation factor. According to embodiments, the membrane may be asymmetrical, such that the filtering properties are different depending on the direction of gas flow. This may be based, for instance, on the permeation behaviour seen in CO₂/CH₄ mixtures in the cellulose acetate membrane system, for example.

Typical filtration requires use of a compressor with outputs typically in the range 5-15 bar pressure. However, according to embodiments, the biogas storage volume provides sufficient space for a large surface area of selectively permeable membrane, such as 500-10,000 m². Such a configuration offers a very low flow resistance through which the biogas constituents are enriched. Coupled with the fact that, in some instances, time is not necessarily a driving factor, molecular mass and molecule size provide a a natural selective permeation process across the membrane. The enrichment process may still be enhanced through a pressure differential, but at a significantly lower levels, for instance in the 10-300 mB range.

The stretching of the membrane under moderate pressure could be used advantageously where changing or controlling the pressure in any one of the storage volumes is used to tune the separation factor between the gas constituents, and thereby used to speed up or slow down the separation process for optimization. For example, the separation factor of a particular membrane may be different from batch to batch due to variations in manufacturing process variables between manufacturers. By adjusting the relative pressure in each volume the membrane can be stretched or allowed to relax increasing or decreasing pore size respectively. As the separation factor may be strongly reliant of the average pore size, it can be adjusted accordingly. This could also solve issues of clogging due to moisture vapour, particulates etc. According to embodiments, if clogging becomes an issue, the pressure could be increased stretching the membrane and its pores to a point where the clogging is alleviated and the H₂O and particulates pass through the membrane. This step could be introduced into a biogas refining operating procedure to extend the life of the membrane, including as a step in any of the anaerobic digester operations described herein. In certain aspects, a method of operating an anaerobic digester can include a step of self-cleaning through membrane stretching, e.g., by pressurized flow through one or more membranes.

According to embodiments, a batch process is provided with continual refinement by moving gases through successive passes through the system. In certain aspects, the gas may move through continuously. In certain aspects, the gas is at low pressure (e.g., using a blower). In certain aspects, a compressor is not used.

Depending on the purity required for a particular purpose, the gas can be taken out at the appropriate space within the stack. The process of ultra-low pressure diffusion-enhanced molecular refinement is slow, taking place over days. This is accounted for by keeping the volume of the biogas storage container 104 sufficiently large so as to be able to hold the biogas generation capacity of the anaerobic digester at least over a similar period.

In some embodiments, biogas that is at a higher level in the biogas storage container 104 may be passed down to a lower level or vice versa. For instance, biogas in space 108 and/or space 108a may be fed back to space 106. The gas may be fed back by using a low-pressure pump (which may be solar powered). Doing this causes the biogas to go through a gravitation-assisted diffusion process another time. Such additional refinement can improve the separation of methane and carbon dioxide. Because the biogas may be stored in the biogas storage container 104 for an extended period of time (e.g., days and/or weeks) before being retrieved and processed by a mobile processing plant, there is ample time to allow for additional refinement by pumping the gas at a higher level to a lower level of the biogas storage container 104 or vice versa.

Typical ratios of methane to carbon dioxide in raw biogas are about 60:40. In embodiments, after an extended period (e.g. to allow for the ultra-low pressure diffusion-enhanced refinement due to the semi-permeable membrane 110), this ratio will rise to 85:15 in the upper space (i.e. first space 108) and reduce to 35:65 methane to carbon dioxide in the lower space (i.e. second space 106). The membrane material may be optimized to increase the first ratio (that is, the methane-to-carbon-dioxide ratio in the second space 106) and reduce the second ratio (that is, the methane-to-carbon-dioxide ratio in the first space 108) over the likely primary refinement and collection period. For subsequent processing by the mobile processing plant, it is advantageous to have the first space 108 be CH₄ enriched (e.g. greater than 60:40 ratio, such as an 85:15 ratio or higher) and the second space 106 be CH₄ depleted (e.g. less than 60:40 ratio, such as a 35:65 ratio or lower).

The anaerobic digester 100 operates more efficiently compared to other systems. For instance, a degree of heating is provided by the greenhouse effect enabled by the use of a clear, transparent cover 112 (such as a plastic roof). The cover 11 also stops rainwater from cooling the upper surface of the biogas storage container 104. The digester's size can be varied to allow for different anaerobic digestion timescales. For instance, the digester can be made sufficiently large to allow for a very slow, long anaerobic digestion process. This can result in a more efficient conversion of biomass material to biogas. For example, a retention period of 200 days can be accommodated. Such a period aligns with the annual digestate management period for a typical dairy farm, whereas the standard industrial scale anaerobic digester would have a retention period of about 40 days. The digester design also removes the requirement for stirring systems, which can be complicated, unreliable, and power hungry. According to some embodiments, one or more of the permeable membranes is removable, such that they can be periodically cleaned or replaced.

Additionally, much of the gas refinement is carried out slowly, at low pressure, enhanced by diffusion through the use of a simple semi-permeable membrane before final refinement using a fast, high-pressure system. This two-stage refinement improves the overall energy efficiency of the gas refinement process. Additionally, in embodiments, there is no investment required by the owner of the anaerobic digester for biogas processing and liquefaction equipment. Because the digester can be used with the mobile biogas processing plant, the costs required for such processing can be spread to a purchaser of the excess biogas, thereby enabling small- to mid-sized farms the ability to economically utilize anaerobic digestion. Further, because the anaerobic digester provides other benefits (discussed above) in addition to any revenue that may result from sale of excess biogas, a farm owner looking to install this equipment need not focus solely on the gas production rate to determine the value of investment in this equipment. Any revenue from gas sales would be a bonus on top of the other available benefits. Also, the investment case for the biogas processing plant is also different. The costs of the biogas processing plant, because it is mobile, may be spread out among the purchasers of biogas or the multiple farms that may utilize a single mobile biogas processing plant. Additionally, because the plant is mobile, it can effectively be in use continuously (apart from travel time), which also improves the investment case for such a plant.

A mobile biogas processing plant is now described. The mobile biogas processing plant may also include liquefaction and storage equipment.

In order to make anaerobic digestion feasible for small- to mid-sized farms, it is important to be able to process, store, and collect the partially refined biogas products from time to time. In order to do so, an ultra-compact, efficient, and self-powering mobile biogas separation and liquefaction processing plant is provided. Such a processing plant needs to be able to handle the large volumes of gas required in a short period of time in order to make the process work economically.

A biogas processing and liquefaction system was introduced in the Mann Patent. Improvements relative to that system are described here, although it is to be noted that such modifications are generally applicable to other biogas processing and liquefaction systems.

In embodiments, the mobile biogas processing plant operates at high pressure, such as from about 200 bar to about 300 bar. The size of the processing plant is thereby reduced, meaning that the size of the various subsystems such as cold vessels, heat exchangers, and pipework may also be correspondingly reduced. For example, compared to a system operating at about 20 bar (as conventional biogas processing systems do), a system operating at about 200 bar may take up 1/10th of the volume. This shift to high-pressure operation, therefore, allows for ultra-compact components while still allowing high throughput. While high-pressure operation is used in some embodiments, other embodiments may utilize a lower pressure setup.

Additionally, and according to some embodiments, by the time that the mobile biogas processing plant arrives to collect the biogas from the anaerobic digester 100, the biogas has already been partially refined. By carrying out a preliminary enrichment of the methane level in the biogas prior to the final enrichment, the overall efficiency of the process is improved, which can lead to a more compact biogas processing plant e.g. through the reduction in size for processing components (such as compressors). Also, the power used to drive the compressor that compresses the partially enriched biomethane from the upper space of the biogas storage container 104 may be generated using an engine configured to run on the partially enriched carbon-dioxide biogas taken from the lower space of the biogas storage container 104. As CO₂ is inert, the air/biogas mixture feed can be adjusted using well-known techniques to allow the engine to run normally. The partially enriched (and therefore lower value) carbon-dioxide biogas may also be used to provide power for other components of the mobile biogas processing plant. This gas would otherwise have to have a secondary process to remove any remaining CH₄ before being released to the atmosphere, in order to minimize any fugitive emissions.

Because the generator is a low-cost bulky item, each farm can have a generator installed on it, e.g. near the site of the anaerobic digester 100. This further reduces the size of the biogas processing plant, and also has the further advantage of providing the farm with power and heat when they are needed, which can be a useful cost saving for the farm. As an alternative, the engine that powers the vehicle transporting the mobile biogas processing plant can also be used to drive an onboard generator or compressor via a power take-off (PTO) point in the transmission or drive train. This also ensures that the mobile biogas processing plant will have access to the generator when needed.

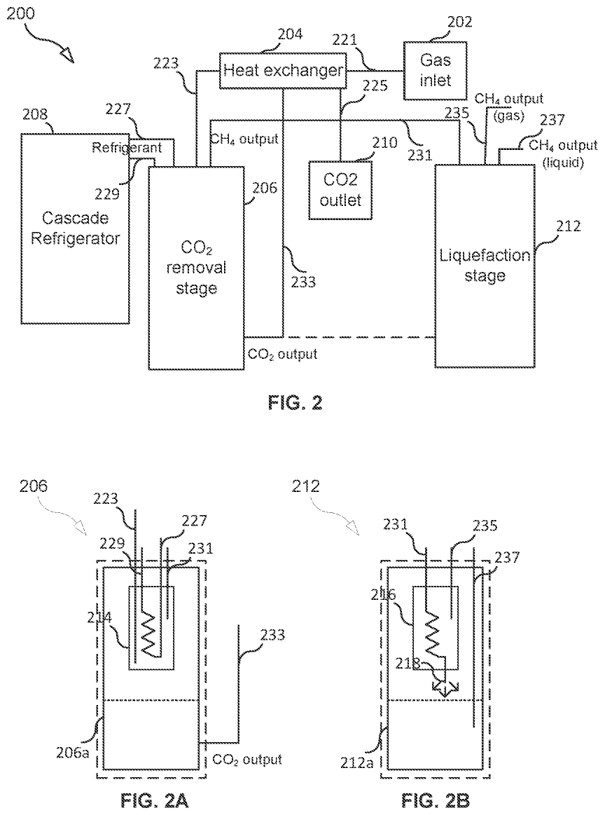

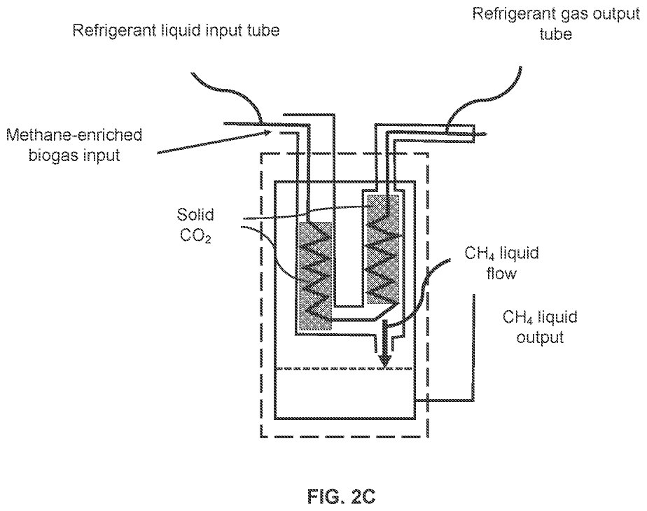

According to embodiments, a vehicle (e.g., a truck or tractor) is provided that comprises the mobile biogas processing plant, such as illustrated with respect to FIGS. 2, 2A, 2B, and 2C.

Embodiments also provide for a power-efficient super-compact methane enrichment and liquefaction system. In order to minimize the power required to achieve cryogenic separation and subsequent methane liquefaction, a triple stage cooling process may be used.

Embodiments may also utilize other sources of providing cooling, either as an alternative to, or complementarily with, the any of the techniques disclosed herein. For example, embodiments may use a sacrificial cryogenic liquid such as liquid nitrogen or liquid air, which both can be easily transported to the anaerobic digester site and used as a convenient source of cooling. Any cryogenic gas that is liquid at a colder temperature than methane could also be used here. Gasses such as argon, oxygen, or hydrogen are also appropriate. Because nitrogen and liquid air are abundant and relatively cheap and easy to create, using these gasses may be more advantageous than others. Using cryogenic liquids in this manner, combined with a heat exchanger for example, may further enable embodiments to replace the need for complicated refrigeration hardware, thereby reducing cost and power requirements of the mobile gas processing system. Another advantage of this approach is that the sacrificial cryogenic liquid can be produced elsewhere, e.g. using renewable energy such as wind, hydro or solar and thereby rendering it cheap and also zero carbon.

The system is also able to optimize the financial return of the outputs or the logistics or commercial sales requirements, as the system can be reconfigured remotely through the opening or closing of valves to pass the products through in a different sequence to supply high-purity methane as a liquid with maximum energy efficiency. Alternatively, as a high-pressure ambient temperature gas for supply as compressed methane, with the cold being redirected to the biogas input via a heat exchanger to further reduce power requirement. Finally, the output from the liquefaction stage is very pure methane that can be compressed and sold at a premium. The system can also supply high-purity liquid CO₂, dry ice, or CO₂ gas if desired. Alternatively, it can recycle the remaining cold in the CO₂ output to further improve the energy efficiency and purity of the methane liquefaction process through pre-cooling the incoming biogas. That is, the energy that has been expended to cool the CO₂ (e.g. to approximately −60° C.) can be mostly recovered (or “recycled”) by passing the CO₂ through a heat exchanger to cool the incoming biogas stream.

The CO₂ enriched biogas from the anaerobic digester 100 may be separately extracted from the methane enriched biogas. For instance, if the purity of the CO2 enriched biogas reaches a certain level (e.g. down to less than or equal to 0.1% methane), then the gas may be extracted from the digester 100 into a tank for storing CO₂. This may be done when the mobile biogas processing plant arrives to process the biogas. Alternatively, or in addition, there may be equipment (such as one or more tanks and a compressor) located near the digester 100 for storing the CO₂. This may be done automatically, based on circuitry and/or processing elements that can detect when the purity of the gas reaches a threshold level, and then extract the gas, compress it (e.g. to about 50 bar), and then store it in appropriate cylinders or tanks for storing carbon dioxide. There may be, for instance, a gauge on or nearby the digester 100 indicating the purity of the carbon dioxide and/or an amount of carbon dioxide present.

FIG. 2 illustrates an exemplary biogas separation and methane liquefier 200, according to some embodiments. FIG. 2A illustrates an exemplary CO₂ removal unit (e.g., cold box) 206 according to some embodiments. FIG. 2B illustrates an exemplary liquefaction unit (e.g., Joule-Thompson unit) 212 according to some embodiments. FIG. 2C illustrates an exemplary CO₂ removal and liquefaction unit according to some embodiments. The biogas separation and methane liquefier 200 is now described with reference to FIGS. 2, 2A, 2B, and 2C.

The biogas mixture (e.g. the methane enriched biogas, such as from one or more of the methane-enriched spaces of the anaerobic digester 100) is optionally first compressed by a compressor (not shown) to a processing pressure (e.g. between 100 bar and 300 bar), filtered by one or more filters (not shown), and then fed to the gas inlet 202. As a general matter, the lower the processing pressure, the less energy is required for liquefaction, while the higher the processing pressure, the easier it is to separate the carbon dioxide and methane (e.g. because there is more separation in phase diagrams for allowing the carbon dioxide to become a liquid while the methane remains a gas). The processing pressure could be as low as 30 bar, and may be higher than 300 bar. Also, the size of components may generally be made smaller as the pressure increases, e.g. due to the volume of gas decreasing at higher pressure. As noted, in exemplary embodiments, the processing pressure is around 100 bar to 300 bar. The biogas entering at inlet 202 will be, in some embodiments, approximately 85% methane and approximately 15% carbon dioxide, from about 100 bar to 300 bar, and about 20° C. (or whatever temperature the biogas is at after exiting the anaerobic digester 100). In embodiments, the biogas may be pre-processed, such that one or more of compression and/or filtering are not required.

From gas inlet 202, the biogas mixture passes through piping 221 to a heat exchanger 204 and then through piping 223 to a CO₂ removal unit (e.g., cold box) 206. Heat exchanger 204 may, in some embodiments, be cooled by CO₂ (e.g. at around −60° C.) before the biogas flows through piping 223 to enter the cold box 206. The CO₂ (e.g., in liquid form) that cools the heat exchanger 204 may be supplied by the removal unit 206 by piping 233 (in which case it will be at approximately the temperature of the cold box), prior to the liquid CO₂ exiting the system by piping 225 to the CO₂ outlet 210. Such CO₂ can also be provided to the liquefaction unit 212 in some instances, as a source of cold.

Inside the removal unit 206, there may be a second heat exchanger 214 (see, e.g., FIG. 2A), which may be cooled by a high power cascade refrigerator 208 driving a circuit of cooled refrigerant (or other cooling method, e.g. a cryocooler or liquid cryogen). The cooling by the cascade refrigerator may cool the cold box 206 to a temperature appropriate for causing the carbon dioxide to liquefy (or drop out as a solid) while maintaining the methane as a gas. The precise temperature will depend on the processing pressure. For instance, at pressures of about 100 bar-300 bar and temperatures of about −40° C. to −60° C., methane is a gas and CO₂ condenses to form liquid. In embodiments, the temperature that the cold box 206 is cooled to may be approximately from about −40° C. to −60° C., and in embodiments may be approximately −60° C. Refrigerator 208 is coupled to the cold box 206 by piping 227 and 229, which carries refrigerant into and out of the cold box 206, respectively.

According to embodiments, the methane is cooled but remains a gas as it passes over the heat exchanger 214, whereas the CO₂ condenses to a liquid (or, in some embodiments, a solid) and falls to the bottom 206a of the cold box 206. The extracted CO₂ may then exit cold box 206 by piping 233. In some embodiments, solid CO₂ may be retained in solid form until a batch of biomethane has been refined or the box is full, when the system may be shut down, the equipment warmed, and the CO₂ may be removed in either gaseous or liquid form. As noted above, it may in some embodiments first pass through the heat exchanger 204 in order to take advantage of the fact that liquid CO₂ is cooled by the cold box (e.g., to approximately −60° C.). Doing this can save considerable energy requirements, because the cascade refrigerator 208 will not need to cool the gas entering the cold box as much in such a case. When the CO₂ leaves the heat exchanger 204 by piping 225 and reaches the outlet 210, it may have an approximate temperature of around 20° C. (or approximately whatever temperature the biogas entering the heat exchanger 204 has), and be at about 100 bar-300 bar. The cold box 206 is insulated (insulation shown by dashed lines around cold box 206) to conserve cold and reduce the cooling power requirement.

The now cold, but still pressurized methane, passes by piping 231 to the liquefaction unit (e.g., Joule-Thompson unit) 212. The liquefaction unit may also serve as a storage unit. However, the system 200 may include additional methane storage units (not illustrated), which can be removable as needed. When passing through piping 231, the gas is approximately 99% pure methane, with around 1% carbon dioxide, is still at around 100 bar-300 bar, and is cooled due to the cold box 206 (e.g. to approximately −60° C.). In the examples of FIG. 2B, the Joule-Thompson unit 212 is where the liquefaction stage of the process for the methane gas takes place. The unit 212 is insulated (shown in dashed lines), which can help conserve cold and reduce the cooling power requirement. The pressurized methane passes through a heat exchanger 216 (see FIG. 2B) within unit 212. The heat exchanger 216 may be cooled by the outgoing low pressure methane (that passes through piping 235), causing the pressurized methane to cool further before passing through an orifice 218 (such as a Joule-Thompson orifice), where the methane finally cools to a low enough temperature to liquefy. The methane entering through piping 231 is at about the pressure of the methane in the cold box 206, e.g. approximately 100 bar to 300 bar in some embodiments. The pressure as the gas passes through the orifice 218 reduces to a low pressure, e.g. about 1 bar. The methane is cooled by the heat exchanger 216 to a temperature at which the methane will liquefy. This will depend on the pressure after the gas passes through the orifice 218, but in some embodiments, the temperature may be approximately −161° C. or lower. If the temperature is too cold, the methane may solidify, which would block the output pipework. Therefore the temperature is preferably cold enough to cause the methane to become liquid, but not too cold to solidify the methane. The liquefied methane falls to the bottom 212a of the unit 212, where it is at an approximate temperature in some embodiments of about −161° C. Because the methane is already cold and is at high pressure when it enters unit 212, the liquefaction fraction will be high, typically 70%-80%, resulting in a very efficient process. That is, most of the methane will liquefy and exit by piping 237 as liquid methane, for instance at retrieval or when moved to on-board storage. Some of the methane, however, will remain in gaseous form, and will exit via piping 235 as gas, at a lower pressure of about 1 bar. At this point, both the liquid and gaseous methane may be very pure, in embodiments more than 99% pure methane.

The Joule-Thompson unit 212 just described is an exemplary mechanism for liquefying methane gas. In some embodiments, a cryocooler, Brayton cycle device, or other device for liquefying methane may be used. Further, while the description above noted that the cold box is configured to liquefy CO₂ gas but not methane gas, in embodiments the cold box may be configured to liquefy and/or solidify CO₂ gas but not methane gas.

For high levels of methane refinement where the CO₂ makes up a small fraction of the total volume (such as approximately 1-10%), the CO₂ can conveniently be removed as a solid without requiring equipment (such as a cold box or heat exchanger) that is bulky or too large for being used as part of a mobile biogas processing plant. The methane can then be simultaneously removed as a liquid at low pressure. By sizing the heat exchanger appropriately, this can occur within a common liquefaction and low-pressure CO₂ removal unit (e.g., cold box enclosure), such as shown in FIG. 2C. In certain scenarios where there is limited power availability at the site to drive a compressor, this can provide a more energy-efficient solution and can be further enhanced through the use of a low-cost sacrificial cold source such as an inert liquid cryogen, for example liquid nitrogen. Where appropriate this can conveniently be brought to the site as a liquid in sufficient quantity to carry out the gas processing required for the period in question. In some embodiments, gas inlet 202 is adapted for receiving CO₂ enriched biogas. In embodiments, the methane-enriched and CO₂ enriched inlets comprise a single inlet 202.

As well as the source of cold being a sacrificial cryogenic liquid, where convenient it could also be a mechanical cooler used to liquefy air at the site or alternatively a close cycle refrigeration circuit. Whichever source is used, it must provide sufficient cooling for both phase changes in the CO2 gas to solid and the methane gas to liquid in embodiments. Where the refrigerant is a sacrificial cryogenic liquid such as liquid nitrogen or liquid argon that has a boiling point lower than the freezing point of methane care must be taken to ensure that the methane liquefaction process temperature is maintained above the freezing point of methane at the process operating pressure otherwise solid methane will form causing blockages in the heat exchanger path. At atmospheric pressure methane freezes at approximately −182° C. which is above both the boiling point of liquid nitrogen and liquid argon. A safe liquefaction operation temperature can conveniently be achieved by holding the sacrificial cryogenic liquid at a higher than atmospheric pressure via a pressure release valve. This also has the advantage of providing a failsafe system ensuring that its boiling point is maintained above that of the freezing point of methane without the need for active control. For liquid nitrogen for example a pressure of 5 bar would maintain a boiling point of approximately 172° C. ensuring that the methane gas stream never freezes.

According to embodiments, solid CO₂ may be used to improve the liquefaction process in stage 212. For instance, a refrigerant liquid can be introduced to cause solid-form CO₂ buildup in stage 212, which can in turn provide a source of cold for liquefaction of the methane. As such, a liquefaction stage 212 may comprise a refrigerant liquid input and output, as shown in FIG. 2C. In some embodiments, liquid refrigerant may be provided in an outer region of the stage 212, which is separate from the liquefaction chamber, as illustrated with the dashed-line box of FIG. 2C. In embodiments, the dashed-line box may instead represent an insulation layer. In some embodiments, and as illustrated in FIG. 2C, a sacrificial refrigerant liquid (e.g., liquid nitrogen or liquid air) can be introduced by a flexible tube or pipe. Similarly, it may be extracted (e.g., in gas form) via an outlet tube or pipe. In some embodiments, and as shown in FIG. 2C, the refrigerant tube or pipe may be located within the input tube or pipe of the biogas (e.g., methane-enriched biogas). That is, the liquefaction stage may use a tube-in-tube (or pipe-in-pipe, or tube-in-pipe) arrangement with cold liquid flowing within the biogas flow path (or vice-versa). This arrangement may beneficially cause a build-up of solid CO₂ in the path of the biogas, which can have benefits for both purification and cooling of the biogas. That is, biogas may flow through solid or liquid-form CO₂ extracted from biogas or generated from a sacrificial source. According to embodiments, the refrigerant liquid may be from an extraction stage of the unit, or retrieved from on-board storage of a mobile unit (e.g., on a truck).

According to embodiments, the whole system is compact enough to fit onto the back of a small truck and be powered from a small methane powered engine. In certain aspects, the biogas can be processed while the system is in transit. For instance, biogas may be processed while a vehicle housing system 200 is travelling between one or more digesters (e.g., digesters 100), or between a digester and a central hub or storage location, as illustrated in connection with FIG. 3.

Logistical solutions for managing multiple installations of anaerobic digesters and managing the distribution of one or more mobile biogas processing plants among the multiple installations are now described.

FIG. 3 illustrates an exemplary system 300 according to some embodiments. As shown, installations 302-308 may be communicatively coupled with a logistics coordination center 314. Logistics coordination center may also be communicatively coupled to one or more mobile biogas processing plants 310 and one or more grids 312. Each installation 302-308 may have one or more anaerobic digesters 100 associated with it. The installations may include farms such as dairy cow farms, where the installations include covered slurry lagoons where cow manure or other farm-related biomass is fed into the anaerobic digesters 100, and/or grasslands where grass clippings and related biomass is fed into the anaerobic digesters 100. Each installation may also include one or more sensors (e.g. measuring biogas level, concentration, power, etc.). One or more mobile biogas processing plants 310 may serve the installations, and the logistics coordination center 314 may monitor the one or more sensors form the different installations and coordinate delivery of the mobile biogas processing plants 310 based at least in part on the sensor readings. Mobile processing plants 310 may each include, for example, biogas separation and methane liquefier 200.

Logistics coordination center 314 may also communicate with grid 312, and may coordinate delivery of the mobile biogas processing plants 310 to supply power to grid 312 based on such communication. Logistics coordination center 314 may also receive information about current pricing of biogas, or power provided by the biogas, as well as information regarding contracts or other interest in parties receiving biogas. Logistics coordination center 314 may also receive readings from the mobile biogas processing plants, including their current location, capacity for carrying additional biogas, and additional scheduling related information e.g. indicating their availability for processing operations. Some or all of this information that logistics coordination center 314 receives may be used in making resource allocation decisions.

While a single logistics coordination center 314 is illustrated, it is noted that the logic for the center 314 may be housed together or may be spread out geographically in many different processing nodes. Additionally, there may be multiple logistics coordination centers 314. These multiple centers 314 may be responsible for disjoint sets of installations, overlapping sets of installations, or the same sets of installations. For instance, the multiple centers 314 may communicate with each other regarding their respective capacities and capabilities, and coordinate sharing of resources associated with the respective centers 314. For instance, an association of local farmers, or a political entity, may be responsible for coordinating resources in a given area. That association or political entity, however, may also have agreements with other neighboring associations and political entities regarding sharing of resources. In such a circumstance, it may be that an available mobile biogas processing plant 310 from one association or political entity is sent to service an installation associated with another association or political entity based on need and availability.

Resource allocation decisions made by logistics coordination center 314 may have one or more different strategic goals. For instance, minimization of distance traveled by the biogas processing plants 310 may be one goal; maximizing price received for the biogas may be another goal; and ensuring that each anaerobic digester does not reach a critical capacity level may be another goal. Other goals are also possible, such as ensuring that total time where a certain digester capacity level is exceeded is minimized. Logistics coordination center 314 may attempt to balance one or more of these goals, and may assign priorities to them. Algorithms including machine-learning algorithms may be employed by logistics coordination center 314 to manage resources while ensuring compliance with the different goals.

The combination of a mobile vehicle mounted biogas processing plant with a simple and efficient anaerobic digester incorporating a simple methane and carbon dioxide enrichment membrane system enables profitable access to relatively small sources of biomass such as small farms and recreational establishments that have grass such as golf courses, parks, and sports fields. There are a number of reasons why this is the case.

These reasons include that there is minimal capital investment or infrastructure changes required over and above the existing requirement to manage the waste. The cost of the biogas processing plant is covered by the plant operator. One mobile biogas processing plant can serve a number of sites (e.g., from 4-25 sites) spreading the investment costs accordingly, and a fleet of mobile biogas processing plants can serve a larger number of sites, such as may be distributed across a region (e.g., from 50-100 sites or more located across multiple geographic regions). There is a limited to zero risk that the mobile plant is underutilized, as the gas level at all sites being served by the mobile plant can be remotely monitored and the most efficient processing and collection logistics plan can be calculated using algorithms (such as machine learning algorithms) that take into account parameters such as gas level, quality, distance to site, and sale price. Additionally, the system may be monitored and controlled through the use of the Internet, meaning that there is no need for an operative to remain on site and allowing one operator to manage more than one anaerobic digester system or more than one mobile biogas processing plant. Furthermore, if a system or component fails, then a spare unit can replace it and the failed system or component may be taken to a dedicated workshop, rather than having to repair on site.

While embodiments describe coordination and selection between installations, a single installation may be monitored and independently selected for processing, for instance based on one or more measurement of its biogas.

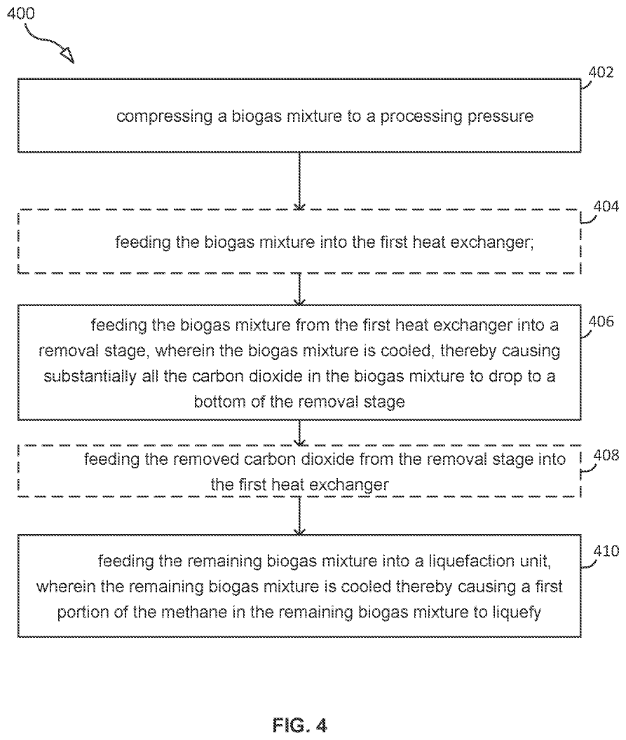

FIG. 4 illustrates a flow chart according to some embodiments. Process 400 is a method for using a mobile biogas processing plant. For instance, method 400 may be performed in connection with one or more system or devices as describe in FIGS. 2 and 3. Process 400 may begin with step 402.

Step 402, comprises compressing a biogas mixture to a processing pressure (e.g., in a range of from about 100 bar to about 300 bar).

Step 404, which may be optional in some embodiments, comprises feeding the biogas mixture into the first heat exchanger. As with the initial pressurization of step 402, this step may also be optional. For instance, biogas may be pre-processed to state that is sufficient for separation and liquefaction.

Step 406 comprises feeding the biogas mixture from the first heat exchanger into the CO₂ removal unit (e.g., cold box), wherein the biogas mixture is cooled (e.g., to about at least −60° C.) thereby causing substantially all the carbon dioxide in the biogas mixture to liquefy (or solidify) and drop to a bottom of the cold box.

Step 408, which may be optional in some embodiments, comprises using cold carbon dioxide form step 406 to provide a source of cold for other processing. This can include, for instance, feeding the liquefied carbon dioxide from the cold box into the first heat exchanger.

Step 410 comprises feeding the remaining biogas mixture from the cold box into the liquefaction unit (e.g., Joule Thompson unit), wherein the remaining biogas mixture is cooled (e.g., to about at least −161° C.) thereby causing a first portion of the methane in the remaining biogas mixture to liquefy.

In some embodiments, the biogas mixture prior to being fed into the first heat exchanger is about 85% methane and about 15% carbon dioxide.

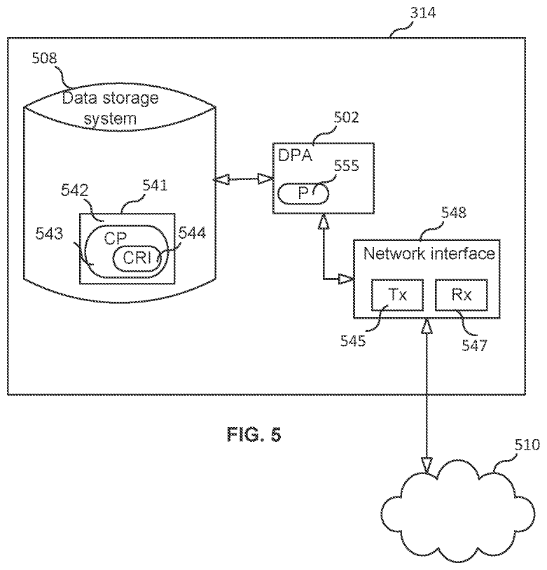

FIG. 5 illustrates a block diagram of an apparatus (such as associated with an anaerobic digester 100, mobile biogas processing plant 310, or logistics coordination center 314) according to some embodiments. As shown in FIG. 5, the apparatus may comprise: processing circuitry (PC) 502, which may include one or more processors (P) 555 (e.g., a general purpose microprocessor and/or one or more other processors, such as an application specific integrated circuit (ASIC), field-programmable gate arrays (FPGAs), and the like); a network interface 648 comprising a transmitter (Tx) 545 and a receiver (Rx) 547 for enabling the apparatus to transmit data to and receive data from other nodes connected to a network 510 (e.g., an Internet Protocol (IP) network) to which network interface 548 is connected; and a local storage unit (a.k.a., “data storage system”) 508, which may include one or more non-volatile storage devices and/or one or more volatile storage devices. In embodiments where PC 502 includes a programmable processor, a computer program product (CPP) 541 may be provided. CPP 541 includes a computer readable medium (CRM) 542 storing a computer program (CP) 543 comprising computer readable instructions (CRI) 544. CRM 542 may be a non-transitory computer readable medium, such as, magnetic media (e.g., a hard disk), optical media, memory devices (e.g., random access memory, flash memory), and the like. In some embodiments, the CRI 544 of computer program 543 is configured such that when executed by PC 502, the CRI causes the apparatus to perform steps described herein (e.g., steps described herein with reference to the flow charts). In other embodiments, the apparatus may be configured to perform steps described herein without the need for code. That is, for example, PC 502 may consist merely of one or more ASICs. Hence, the features of the embodiments described herein may be implemented in hardware and/or software.

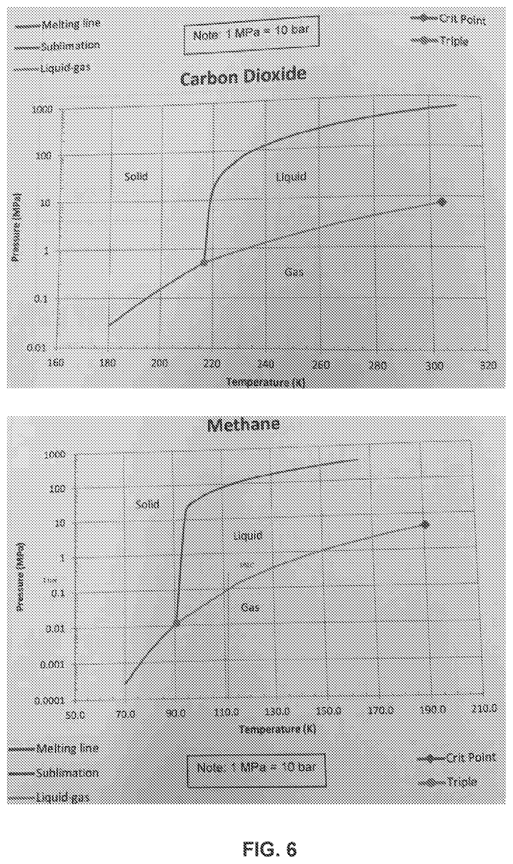

FIG. 6 shows the phase diagrams for both methane and carbon dioxide. As discussed above, the temperature appropriate in different phases of the liquefaction process may depend on the pressure of the gases. The diagrams generally show when methane and carbon dioxide are expected to be in a liquid, solid, or gaseous stage, for a given pressure and temperature.

While various embodiments of the present disclosure are described herein, it should be understood that they have been presented by way of example only, and not limitation. Thus, the breadth and scope of the present disclosure should not be limited by any of the above-described exemplary embodiments. Moreover, any combination of the above-described elements in all possible variations thereof is encompassed by the disclosure unless otherwise indicated herein or otherwise clearly contradicted by context.

Additionally, while the processes described above and illustrated in the drawings are shown as a sequence of steps, this was done solely for the sake of illustration. Accordingly, it is contemplated that some steps may be added, some steps may be omitted, the order of the steps may be re-arranged, and some steps may be performed in parallel.

1-22. (canceled)

23. A mobile biogas processing plant comprising:
a methane input for receiving methane-enriched gas;
one or more carbon dioxide, CO₂, removal stages configured to remove CO₂ from the methane-enriched gas, and
a liquefaction stage configured to generate liquid methane from the methane-enriched gas.

24. The mobile biogas processing plant of claim 23, further comprising:
a CO₂ input for receiving CO₂-enriched gas, wherein at least one of the CO₂ removal stages is configured to generate liquid or solid CO₂ from the CO₂-enriched gas.

25. The mobile biogas processing plant of claim 23, wherein at least one of the CO₂ removal stages and the liquefaction stage are a single combined stage.

26. The mobile biogas processing plant of claim 23, wherein at least one of the CO₂ removal stages is configured to operate using liquid or solid CO₂ extracted from the methane-enriched biogas.

27. The mobile biogas processing plant of claim 23,
wherein the liquefaction stage is configured to operate using liquid or solid CO₂ extracted from the methane-enriched or CO₂-enriched biogas, or
wherein the liquefaction stage comprises a sacrificial cooling liquid input having a tube-in-tube, tube-in-pipe, or pipe-in-pipe arrangement, and is configured to operate using sacrificial cooling material.

28. The mobile biogas processing plant of claim 23, wherein a least one of the CO₂ removal stages comprises one or more of a heat exchanger, refrigeration unit, or sacrificial cooling liquid.

29. The mobile biogas processing plant of claim 23, wherein the liquefaction stage comprises one or more of a Joule Thompson unit, cryocooler, Brayton cycle device, refrigeration circuit, or sacrificial cooling liquid.

30. The mobile biogas processing plant of claim 29, further comprising:
a storage unit for the sacrificial cooling material.

31. The mobile biogas processing plant of claim 23, wherein the methane inputs is coupled to an anaerobic digester comprising at least one semipermeable membrane that is selectively permeable between CO₂ and methane.

32. The mobile biogas processing plant of claim 23,
wherein the mobile biogas processing plant comprises a vehicle and is transportable to a remote site.

33. The mobile gas processing plant of claim 23, wherein the processing plant comprises liquid nitrogen or liquid air storage that is removable and replaceable with a replacement unit.

34. The mobile biogas processing plant of claim 23, further comprising:
a liquid methane storage unit coupled to an output of the liquefaction stage, wherein the methane storage unit is removable and replaceable with a replacement unit.

35. A method for using a mobile biogas processing plant, the method comprising:
receiving methane-enriched gas;
removing carbon dioxide, CO₂, from the methane-enriched gas; and
generating liquid methane from the methane-enriched gas after the removal,
wherein one or more of the removing or generating are performed on a vehicle.

36. The method of claim 35, further comprising:
receiving CO₂-enriched gas; and
generating liquid or solid CO₂ from the CO₂-enriched gas.

37. (canceled)

38. A mobile biogas processing plant comprising:
a methane input for receiving methane-enriched gas;
a compressor coupled to the methane input and configured to compress methane gas;
a first heat exchanger coupled to the compressor;
a CO₂ removal stage coupled to the first heat exchanger, wherein the removal stage is configured to remove CO₂ from an input gas in solid or liquid form, but not solidify of liquefy methane; and
a liquefaction unit coupled to the removal stage, wherein the liquefaction unit is configured to liquefy methane gas.

39. The mobile gas processing plant of claim 38, wherein the removal stage comprises a cold box and the liquefaction stage comprises a Joule Thompson unit, sacrificial liquid input, or cryocooler.

40. The mobile biogas processing plant of claim 38,
wherein the mobile biogas processing plant comprises a vehicle and comprises a methane storage, and
wherein the methane storage is removable and replaceable such that the methane storage can be removed and replaced when full or nearly full with a replacement unit.

41. The mobile biogas processing plant of claim 38, wherein the methane inputs is coupled to an anaerobic digester comprising at least one semipermeable membrane that is selectively permeable between CO₂ and methane.

42. The mobile biogas processing plant of claim 38, further comprising:
a CO₂ input for receiving CO₂-enriched gas,
wherein the CO₂ input is coupled to an anaerobic digester, and
wherein the compressor is configured to be powered by CO₂-enriched gas received from the CO₂ input.

43-56. (canceled)
